(19) United States
(12) Patent Application Publication
(10) Pub. No.: US 2021/0205272 A1
(43) Pub. Date: Jul. 8, 2021

(54) DOSING REGIMENS FOR TREATMENT OF SOLID TUMORS HAVING ONE OR MORE GENETIC ALTERATIONS IN FGFR1, FGFR2, AND/OR FGFR3

(71) Applicant: DEBIOPHARM INTERNATIONAL, S.A., Lausanne (CH)

(72) Inventors: Claudio ZANNA, Lausanne (CH); Keith T. FLAHERTY, Cambridge, MA (US); Hiroaki TANAKA, Lausanne (CH); Anna POKORSKA-BOCCI, Denens (CH); Franck BRICHORY, Bonne (CH)

(21) Appl. No.: 17/055,886

(22) PCT Filed: May 15, 2019
(86) PCT No.: PCT/IB2019/054043

§ 371 Date: Nov. 16, 2020

Related U.S. Application Data
(60) Provisional application No. 62/672,292, filed on May 16, 2018.
(60) Provisional application No. 62/672,295, filed on May 16, 2018.
(60) Provisional application No. 62/796,497, filed on Jan. 24, 2019.

(51) Int. Cl. A61K 31/4184; A61K 9/20; A61P 35/00

(52) U.S. Cl. CPC A61K 31/4184; A61P 35/00; A61K 9/20

(57) ABSTRACT

Methods of administering Compound A, or pharmaceutically acceptable salt thereof, for the treatment of solid tumors having one or more genetic alterations in FGFR1, FGFR2 and/or FGFR3 are provided.

### CROSS-REFERENCE TO RELATED APPLICATIONS

This application claims priority to U.S. Provisional Application No. 62/672,292, filed May 16, 2018, U.S. Provisional Application No. 62/672,295, filed May 16, 2018, and U.S. Provisional Application No. 62/796,497, filed Jan. 24, 2019; which applications are each incorporated herein by reference in their entireties.

### FIELD OF THE INVENTION

The field of the invention generally relates to methods of treating a human patient having a solid tumor with one or more genetic alterations of Fibroblast Growth Factor Receptor 1 (FGFR1), Fibroblast Growth Factor Receptor 2 (FGFR2) and/or Fibroblast Growth Factor Receptor 3 (FGFR3) comprising administering to the patient, in need thereof, 5-amino-1-(2-methyl-1H-benzo[d]imidazol-5-yl)-1H-pyrazol-4-yl 1H-indol-2-yl ketone (CAS No. 1265229-25-1, also known as Debio 1347 and CH5183284, hereinafter referred to as Compound A), or pharmaceutically acceptable salt thereof.

### BACKGROUND OF THE INVENTION

Cancer (e.g., a solid tumor) is one of the leading causes of death in the developed world, with over one million people diagnosed with cancer and 500,000 deaths per year in the United States alone. Overall it is estimated that more than 1 in 3 people will develop some form of cancer during their lifetime.

There is a need for a means to determine which patients are likely to respond to treatment with an anti-cancer compound, such as Compound A or its pharmaceutically acceptable salts, and for incorporating such determinations into effective treatment regimens for patients with tumors (e.g., solid tumors).

### BRIEF SUMMARY OF THE INVENTION

Methods of administering Compound A, or pharmaceutically acceptable salt thereof, to a human patient having a solid tumor in an amount corresponding to an amount between about 40 mg and about 150 mg of Compound A, as its free base, daily are provided herein. In certain embodiments, the method is for treating a human patient having a solid tumor with one or more genetic alterations in FGFR1, FGFR2, and/or FGFR3 comprising administering to the patient, in need thereof, Compound A, or pharmaceutically acceptable salt thereof. Methods for treating a human patient having a solid tumor with one or more genetic alterations in FGFR1, FGFR2, and/or FGFR3, wherein the method comprises the steps of: (a) obtaining or having obtained a biological sample from the patient; (b) performing or having performed an assay on the biological sample to determine if the patient has one or more genetic alterations in FGFR1, FGFR2, and/or FGFR3; and (c) administering an amount of Compound A, or pharmaceutically acceptable salt thereof, corresponding to an amount between about 40 mg and about 150 mg of Compound A, as its free base, are also provided herein. In certain embodiments, the methods are tissue agnostic, i.e. the solid tumor with one or more genetic alterations in FGFR1, FGFR2, and/or FGFR3 is of any histological type.

The present disclosure also relates to Compound A, in a daily amount between about 40 mg and about 150 mg as its free base, or a pharmaceutically acceptable salt thereof, for use in the treatment of a human patient having a solid tumor, and to the use of Compound A, in a daily amount between about 40 mg and about 150 mg as its free base, or a pharmaceutically acceptable salt thereof, in the manufacture of a medicament for treating a human patient having a solid tumor. In certain embodiments, the solid tumor with one or more genetic alterations in FGFR1, FGFR2, and/or FGFR3 is of any histological type.

In some embodiments, the human patient has a solid tumor with one or more genetic alterations in FGFR1, FGFR2, and/or FGFR3. In some embodiments, the human patient has a solid tumor with one or more genetic alterations in FGFR1, FGFR2, and/or FGFR3 that is an amplification, an activating mutation, a deletion, and/or a fusion/translocation. In some embodiments, the human patient has a solid tumor with one or more genetic alterations that is one or more amplifications in FGFR1, FGFR2, and/or FGFR3. In some embodiments, the human patient has a solid tumor with one or more genetic alterations that is one or more mutations in FGFR1, FGFR2, and/or FGFR3. In some embodiments, the human patient has a solid tumor with one or more genetic alterations that is a deletion in FGFR1, FGFR2, and/or FGFR3. In some embodiments, the human patient has a solid tumor with one or more genetic alterations that is one or more fusions/translocations in FGFR1, FGFR2, and/or FGFR3.

In some embodiments, the human patient has a solid tumor with a genetic alteration that is an amplification in FGFR1.

In some embodiments, the human patient has a solid tumor with a genetic alteration that is an amplification in FGFR2.

In some embodiments, the human patient has a solid tumor with a genetic alteration that is an amplification in FGFR3.

In some embodiments, the human patient has a solid tumor with a genetic alteration that is a mutation in FGFR1.

In some embodiments, the human patient has a solid tumor with a genetic alteration that is a mutation in FGFR2. In some embodiments, the human patient has a solid tumor with one or more mutations in FGFR2 selected from the group consisting of M640I, G384R, T394I, N549K, Y805fs11, and S529C.

In some embodiments, the human patient has a solid tumor with a genetic alteration that is a mutation in FGFR3. In some embodiments, the human patient has a solid tumor with one or more mutations in FGFR3 selected from the group consisting of Y375C, S249C, G561A, V684L, T689M, R248C, and D758N.

In some embodiments, the human patient has a solid tumor with a genetic alteration that is a deletion in FGFR1.

In some embodiments, the human patient has a solid tumor with a genetic alteration that is a deletion in FGFR2. In some embodiments, the human patient has a solid tumor with one or more genetic alterations of exon 5 of FGFR2. In some embodiments, the human patient has a solid tumor with one or more genetic alterations that is a deletion in exon 5 of FGFR2. In some embodiments, the human patient has a solid tumor with one or more genetic alterations that is H167-N173del in exon 5 of FGFR2.

In some embodiments, the human patient has a solid tumor with a genetic alteration that is a deletion in FGFR3.

In some embodiments, the human patient has a solid tumor having a FGFR1 fusion/translocation.

In some embodiments, the human patient has a solid tumor having a FGFR2 fusion/translocation. In some embodiments, the human patient has a solid tumor having a FGFR2 fusion/translocation selected from the group consisting of FGFR2-DDX21, FGFR2-KIAA1217, FGFR2-ROCK1, FGFR2-BICC1, FGFR2-INA, FGFR2-CD44, FGFR2-TACC2, FGFR2-RP11-89K10.1, and FGFR2-ATAD2.

In some embodiments, the human patient has a solid tumor having a FGFR3 fusion/translocation. In some embodiments, the human patient has a solid tumor having a FGFR3-TACC3 fusion/translocation.

In some embodiments, the human patient has a solid tumor having one or more amplifications in FGFR1, FGFR2, and/or FGFR3 and one or more mutations in FGFR1, FGFR2, and/or FGFR3. In some embodiments, the human patient has a solid tumor having one or more amplifications in FGFR1, FGFR2, and/or FGFR3 and one or more fusions/translocations in FGFR1, FGFR2, and/or FGFR3. In some embodiments, the human patient has a solid tumor having one or more mutations in FGFR1, FGFR2, and/or FGFR2 and one or more fusions/translocations in FGFR1, FGFR2, and/or FGFR3. In some embodiments, the human patient has a solid tumor having one or more amplifications in FGFR1, FGFR2, and/or FGFR3 and one or more deletions in FGFR1, FGFR2, and/or FGFR3. In some embodiments, the human patient has a solid tumor having one or more deletions in FGFR1, FGFR2, and/or FGFR3 and one or more fusions/translocations in FGFR1, FGFR2, and/or FGFR3. In some embodiments, the human patient has a solid tumor having mutations in FGFR1, FGFR2, and/or FGFR3 and one or more deletions in FGFR1, FGFR2, and/or FGFR3.

In some embodiments, Compound A is in free base form.

In some embodiments, Compound A is a pharmaceutically acceptable salt. In some embodiments, the pharmaceutically acceptable salt is a malate salt. In some embodiments, the pharmaceutically acceptable salt is a L-malate salt, also called (S)-2-hydroxysuccinate. In some embodiments, the pharmaceutically acceptable salt is a hydrochloride salt. In some embodiments, the pharmaceutically acceptable salt is a methane sulfonate salt, such as a methane sulfonate monohydrate salt. In some alternative embodiments, the pharmaceutically acceptable salt is an acetate salt, a succinate salt, a fumarate salt, a maleate salt, a tartrate salt, a citrate salt, a lactate salt, a stearate salt, a benzoate salt or a p-toluenesulfonate salt.

In some embodiments, the human patient has a solid tumor selected from the group consisting of biliary tract cancer, breast cancer, cholangiocarcinoma, urothelial cancer, uterine neoplasm, lung adenocarcinoma, squamous non-small cell lung cancer (sqNSCLC), non-squamous non-small cell lung cancer (NSCLC), gastric cancer, sarcoma, bladder cancer, head and neck cancer, small cell lung cancer, endometrial cancer, esophageal cancer, adenoid cystic carcinoma, gallbladder cancer, colon cancer, thyroid cancer, hepatocellular cancer, prostate cancer, oral cancer, cervical cancer, pancreatic carcinoma, ovarian cancer, and serous carcinoma to the peritoneum. In some embodiments, the cholangiocarcinoma is selected from the group consisting of intrahepatic cholangiocarcinoma, perihilar cholangiocarcinoma, and distal cholangiocarcinoma.

In some embodiments, the amount of Compound A, or pharmaceutically acceptable salt thereof, corresponds to an amount of about 60 mg, about 70 mg, about 80 mg, about 90 mg, about 100 mg, about 110 mg, about 120 mg, about 130 mg, about 140 mg, or about 150 mg of Compound A, as its free base, daily. In some embodiments, the amount of Compound A, or pharmaceutically acceptable salt thereof, corresponds to about 80 mg of Compound A, as its free base, daily. The amount of Compound A, as a pharmaceutically acceptable salt, will be adjusted based on the weight of the salt form to administer the corresponding amount of free base. For example, in some embodiments, the daily amount of Compound A corresponds to about 83 mg, about 96 mg, about 110 mg, about 124 mg, about 138 mg, about 151 mg, about 165 mg, about 179 mg, about 193 mg, or about 206 mg of a malate salt of Compound A. In some embodiments, the daily amount of Compound A corresponds to about 110 mg of a malate salt of Compound A.

In some embodiments, Compound A, or pharmaceutically acceptable salt thereof, is administered orally. In some embodiments, Compound A, or pharmaceutically acceptable salt thereof, is administered in capsule form. In some embodiments, Compound A is administered in tablet form. In some embodiments, the tablet comprises an amount of Compound A, or pharmaceutically acceptable salt thereof, corresponding to about 20 mg, about 30 mg, about 50 mg, about 60 mg, about 80 mg or about 100 mg of Compound A as its free base. In some embodiments, the tablet comprises an amount of Compound A, or pharmaceutically acceptable salt thereof, corresponding to about 80 mg of Compound A as its free base. The amount of Compound A, as a pharmaceutically acceptable salt, will be adjusted based on the weight of the salt form to be included in the tablet. For example, in some embodiments, the tablet comprises an amount of about 28 mg, about 41 mg, about 69 mg, about 83 mg, about 110 mg or about 138 mg of a malate salt of Compound A. In some embodiments, the tablet comprises about 110 mg of a malate salt of Compound A.

In some embodiments, Compound A, or pharmaceutically acceptable salt thereof, is administered as one dose one time per day. In some embodiments, Compound A, or pharmaceutically acceptable salt thereof, is divided into multiple doses that are administered one, two, three, or four times per day.

In some embodiments, Compound A, or pharmaceutically acceptable salt thereof, is administered to the human patient after the patient has fasted for four hours. In some embodiments, the human patient fasts for two hours after Compound A, or pharmaceutically acceptable salt thereof, is administered to the human patient.

In some embodiments, the human patient has relapsed or progressed after administration of at least one prior standard therapy. In some embodiments, the human patient has biliary tract cancer and has relapsed or progressed after administration of at least one prior standard therapy. In some embodiments, the human patient has biliary tract cancer and has relapsed or progressed after administration of gemcitabine-based chemotherapy. In some embodiments, the human patient has urothelial cancer and has relapsed or progressed after administration of at least one prior standard therapy. In some embodiments, the human patient has urothelial cancer and has relapsed or progressed after administration of chemotherapy, an anti-PD1/PDL1 therapy, or a combination of chemotherapy and an anti-PD1/PDL1 therapy. In some embodiments, the chemotherapy is cisplatin-based or carboplatin-based chemotherapy. In some embodiments, the human patient has non-small cell lung cancer and has relapsed or progressed after administration of at least one prior standard therapy. In some embodiments, the human patient has non-small cell lung cancer and has relapsed or progressed after administration of chemotherapy, an anti-PD1/PDL1 therapy, or a combination of chemotherapy and an anti-PD1/PDL1 therapy.

Methods of treating a human patient having a solid tumor with a deletion in exon 5 of FGFR2 comprising orally administering an amount of Compound A, or pharmaceutically acceptable salt thereof, corresponding to an amount between about 40 mg and about 150 mg of Compound A, as its free base, daily are provided herein. In some embodiments, the deletion in exon 5 of FGFR2 is H167-N173del.

Methods of treating a human patient having a solid tumor with one or more gene fusions/translocations in FGFR1, FGFR2, and/or FGFR3 comprising orally administering an amount of Compound A, or pharmaceutically acceptable salt thereof, corresponding to an amount between about 40 mg and about 150 mg of Compound A, as its free base, daily are provided herein. In some embodiments, the human patient has a solid tumor having a FGFR1 fusion/translocation. In some embodiments, the human patient has a solid tumor having a FGFR2 fusion/translocation. In some embodiments, the human patient has a solid tumor having a FGFR2 fusion/translocation selected from the group consisting in FGFR2-DDX21, FGFR2-KIAA1217, FGFR2-ROCK1, FGFR2-BICC1, FGFR2-INA, FGFR2-CD44, FGFR2-TACC2, FGFR2-RP11-89K10.1, and FGFR2-ATAD2. In some embodiments, the human patient has a solid tumor having a FGFR3 fusion/translocation. In some embodiments, the human patient has a solid tumor having a FGFR3-TACC3 fusion/translocation.

Methods of treating a human patient having a solid tumor with one or more gene fusions/translocations in FGFR1, FGFR2, and/or FGFR3 comprising orally administering an amount of Compound A, or pharmaceutically acceptable salt thereof, corresponding to about 80 mg of Compound A, as its free base, daily are provided herein. In some embodiments, the human patient has a solid tumor having a FGFR1 fusion/translocation. In some embodiments, the human patient has a solid tumor having a FGFR2 fusion/translocation. In some embodiments, the human patient has a solid tumor having a FGFR2 fusion/translocation selected from the group consisting in FGFR2-DDX21, FGFR2-KIAA1217, FGFR2-ROCK1, FGFR2-BICC1, FGFR2-INA, FGFR2-CD44, FGFR2-TACC2, FGFR2-RP11-89K10.1, and FGFR2-ATAD2. In some embodiments, the human patient has a solid tumor having a FGFR3 fusion/translocation. In some embodiments, the human patient has a solid tumor having a FGFR3-TACC3 fusion/translocation.

Methods of treating a human patient having a solid tumor with a deletion in exon 5 of FGFR2 comprising orally administering an amount of Compound A, or pharmaceutically acceptable salt thereof, corresponding to about 80 mg of Compound A, as its free base, daily are provided herein. In some embodiments, the deletion in exon 5 of FGFR2 is H167-N173del.

### BRIEF DESCRIPTION OF DRAWINGS

FIG. 1 shows the best target lesion change from baseline (waterfall plot) in (a) all patients or (b) only patients with a confirmed genetic alteration. In this Figure, metastatic follicular dendritic cell sarcoma and high grade sarcomatoid malignant neoplasm are pooled under Sarcoma. Endometrioid adenocarcinoma cervical cancer is re-classified as cervical cancer. Endometrial and uterine are pooled under Uterine neoplasm. Bladder cancer is re-classified as Urothelial carcinoma.

FIG. 2 shows the best target lesion change from baseline in patients with FGFR fusions/translocations irrespective of histology and treated at ≥60 mg (3D plot).

### DETAILED DESCRIPTION OF THE INVENTION

The present invention provides a method of treating a human patient with a solid tumor having one or more genetic alterations in FGFR1, FGFR2, and/or FGFR3 comprising administering to the patient in need thereof, Compound A, or pharmaceutically acceptable salt thereof, in an amount corresponding to an amount between about 40 mg and about 150 mg of Compound A, as its free base, daily.

### I. Definitions

To facilitate an understanding of the present invention, a number of terms and phrases are defined below.

The term “Compound A” refers to 5-amino-1-(2-methyl-1H-benzo[d]imidazol-5-yl)-1H-pyrazol-4-yl 1H-indol-2-yl ketone (CAS No. 1265229-25-1). This compound is also known as Debio 1347 and CH5183284.

The term “pharmaceutically acceptable salt” refers to a form of Compound A that consists of a cationic form of Compound A in combination with a suitable anion, or in the alternative, an anionic form of Compound A in combination with a cation.

The term “respond favorably” generally refers to causing a beneficial state in a subject. With respect to cancer treatment, the term refers to providing a therapeutic effect on the subject. Positive therapeutic effects in cancer can be measured in a number of ways (See, W. A. Weber, J. Nucl. Med. 50:1S-10S (2009)). For example, tumor growth inhibition, molecular marker expression, serum marker expression, and molecular imaging techniques can all be used to assess therapeutic efficacy of an anti-cancer therapeutic. A favorable response can be assessed, for example, by increased progression-free survival (PFS), disease-free survival (DFS), overall survival (OS), or metastasis-free survival (MFS), by complete response (CR), partial response (PR), or, in some cases, stable disease (SD), a decrease in progressive disease (PD), a reduced time to progression (TTP) or any combination thereof.

PFS, DFS, and OS can be measured by standards set by the National Cancer Institute and the U.S. Food and Drug Administration for the approval of new drugs. See Johnson et al, (2003) J. Clin. Oncol. 21(7):1404-1411.

“Progression free survival” (PFS) refers to the time from enrollment to disease progression or death. PFS is generally measured using the Kaplan-Meier method and Response Evaluation Criteria in Solid Tumors (RECIST) 1.1 standards. Generally, progression free survival refers to the situation wherein a patient remains alive, without the cancer getting worse.

“Time to Tumor Progression” (TTP) is defined as the time from enrollment to disease progression. TTP is generally measured using the RECIST 1.1 criteria.

A “complete response” or “complete remission” or “CR” indicates the disappearance of all signs of tumor or cancer in response to treatment. This does not always mean the cancer has been cured. For example, any pathological lymph nodes (whether target or non-target) must have reduction in the short axis to <10 mm. Complete response is generally measured using the RECIST 1.1 criteria. Eisenhauer, E. A., Eur. J. Cancer, 45: 228-47 (2009).

A “partial response” or “PR” refers to a decrease in the size or volume of one or more tumors or lesions, or in the extent of cancer in the body, in response to treatment. Eisenhauer, E. A., Eur. J. Cancer, 45: 228-47 (2009).

“Stable disease” refers to disease without progression or relapse. In stable disease there is neither sufficient tumor shrinkage to qualify for partial response nor sufficient tumor increase to qualify as progressive disease taking as reference the smallest sum diameters while on the study. Eisenhauer, E. A., Eur. J. Cancer, 45: 228-47 (2009).

“Progressive disease” or “disease that has progressed” refers to the appearance of one more new lesions or tumors and/or the unequivocal progression of existing non-target lesions and/or at least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression). Eisenhauer, E. A., Eur. J. Cancer, 45: 228-47 (2009).

“Overall Survival” (OS) refers to the time from patient enrollment to death or censored at the date last known alive. OS includes a prolongation in life expectancy as compared to naive or untreated individuals or patients. Overall survival refers to the situation wherein a patient remains alive for a defined period of time, such as one year, five years, etc., e.g., from the time of randomization or treatment.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already diagnosed with or suspected of having the disorder. In certain embodiments, a subject is successfully “treated” for cancer, e.g., an advanced solid malignancy, according to the methods of the present invention if the patient shows one or more of the following: a reduction in the number of or complete absence of cancer cells; a reduction in the tumor burden; inhibition of or an absence of cancer cell infiltration into peripheral organs including, for example, the spread of cancer into soft tissue and bone; inhibition of or an absence of tumor metastasis; inhibition or an absence of tumor growth; relief of one or more symptoms associated with the specific cancer; reduced morbidity and mortality; improvement in quality of life; reduction in tumorigenicity, tumorigenic frequency, or tumorigenic capacity, of a tumor; reduction in the number or frequency of cancer stem cells in a tumor; differentiation of tumorigenic cells to a non-tumorigenic state; increased progression-free survival (PFS), disease-free survival (DFS), or overall survival (OS), or metastasis-free survival (MFS), complete response (CR), partial response (PR), stable disease (SD), a decrease in progressive disease (PD), a reduced time to progression (TTP), or any combination thereof.

Prophylactic or preventative measures refer to measures that prevent and/or slow the development of a targeted pathological condition or disorder. Thus, those in need of prophylactic or preventative measures include those prone to have the disorder and those in whom the disorder is to be prevented.

As used in the present disclosure and claims, the singular forms “a,” “an,” and “the” include plural forms unless the context clearly dictates otherwise.

It is understood that wherever embodiments are described herein with the language “comprising,” otherwise analogous embodiments described in terms of “consisting of” and/or “consisting essentially of” are also provided.

### II. Methods of Use and Pharmaceutical Compositions

Compound A and its pharmaceutically acceptable salts thereof are useful in a variety of applications including, but not limited to, therapeutic treatment methods, such as the treatment of human patients having a solid tumor with one or more genetic alterations in FGFR1, FGFR2, and/or FGFR3. These methods of treating human patients having a solid tumor with one or more genetic alterations in FGFR1, FGFR2, and/or FGFR3 with Compound A, or pharmaceutical salt thereof, are mostly histology-independent and may thus be applied on solid tumors of any histological type. They may also be called ‘tumor agnostic’, which approach contrasts with classical tumor treatments primarily based on tissue specificity. Methods for treating a human patient having a solid tumor with one or more genetic alterations in FGFR1, FGFR2, and/or FGFR3, comprising administering to the patient, in need thereof, Compound A, or pharmaceutically acceptable salt thereof, in an amount corresponding to an amount between about 40 mg and about 150 mg of Compound A, as its free base, daily, are provided herein.

Methods for treating a human patient having a solid tumor with one or more genetic alterations in FGFR1, FGFR2, and/or FGFR3, wherein the methods comprise the steps of: (a) obtaining or having obtained a biological sample from the patient; (b) performing or having performed an assay on the biological sample to determine if the patient has one or more genetic alterations in FGFR1, FGFR2, and/or FGFR3; and (c) administering an amount of Compound A, or pharmaceutically acceptable salt thereof, corresponding to an amount between about 40 mg and about 150 mg of Compound A, as its free base, are provided herein.

The present disclosure also relates to Compound A, in a daily amount between about 40 mg and about 150 mg as its free base, or a pharmaceutically acceptable salt thereof, for use in the treatment of a human patient having a solid tumor, and to the use of Compound A, in a daily amount between about 40 mg and about 150 mg as its free base, or a pharmaceutically acceptable salt thereof, in the manufacture of a medicament for treating a human patient having a solid tumor.

In some embodiments, the human patient has a solid tumor with one or more genetic alterations in FGFR1, FGFR2, and/or FGFR3 that is an amplification, an activating mutation, a deletion, and/or a fusion/translocation. In certain embodiments, the human patient has a solid tumor with one or more genetic alterations that is one or more amplifications in FGFR1, FGFR2, and/or FGFR3. In some embodiments, the human patient has a solid tumor with one or more genetic alterations that is a mutation in FGFR1, FGFR2, and/or FGFR3. In some embodiments, the human patient has a solid tumor with one or more genetic alterations that is a deletion in FGFR1, FGFR2, and/or FGFR3. In certain embodiments, the human patient has a solid tumor with one or more genetic alterations that is a fusion/translocation in FGFR1, FGFR2, and/or FGFR3.

In some embodiments, the human patient has a solid tumor with a genetic alteration that is an amplification in FGFR1.

In some embodiments, the human patient has a solid tumor with a genetic alteration that is an amplification in FGFR2.

In some embodiments, the human patient has a solid tumor with a genetic alteration that is an amplification in FGFR3.

In some embodiments, the human patient has a solid tumor with a genetic alteration that is at least one mutation in FGFR1.

In some embodiments, the human patient has a solid tumor with a genetic alteration that is at least one mutation in FGFR2. In some embodiments, the human patient has a solid tumor with one or more mutations in FGFR2 selected from the group consisting of M640I, G384R, T394I, N549K, Y805fs11, and S529C.

In some embodiments, the human patient has a solid tumor with a genetic alteration that is at least one mutation in FGFR3. In some embodiments, the human patient has a solid tumor with one or more mutations in FGFR3 selected from the group consisting of Y375C, S249C, G561A, V684L, T689M, R248C, and D758N.

In some embodiments, the human patient has a solid tumor with a genetic alteration that is at least one deletion in FGFR1.

In some embodiments, the human patient has a solid tumor with a genetic alteration that is at least one deletion in FGFR2. In some embodiments, the human patient has a solid tumor with one or more genetic alterations of exon 5 of FGFR2. In some embodiments, the human patient has a solid tumor with one or more genetic alterations that is a deletion in exon 5 of FGFR2. In some embodiments, the human patient has a solid tumor with one or more genetic alterations that is H167-N173del in exon 5 of FGFR2.

In some embodiments, the human patient has a solid tumor with a genetic alteration that is at least one deletion in FGFR3.

In some embodiments, the human patient has a solid tumor having a FGFR1 fusion/translocation.

In some embodiments, the human patient has a solid tumor having a FGFR2 fusion/translocation. In some embodiments, the human patient has a solid tumor having a FGFR2 fusion/translocation selected from the group consisting of FGFR2-DDX21, FGFR2-KIAA1217, FGFR2-ROCK1, FGFR2-BICC1, FGFR2-INA, FGFR2-CD44, FGFR2-TACC2, FGFR2-RP11-89K10.1, and FGFR2-ATAD2.

In some embodiments, the human patient has a solid tumor having a FGFR3 fusion/translocation. In some embodiments, the human patient has a solid tumor having a FGFR3-TACC3 fusion.

The human patient may have a solid tumor exhibiting any combination of genetic alterations. In some embodiments, the human patient has a solid tumor having one or more genetic alterations in each of FGFR1 and FGFR2. In some embodiments, the human patient has a solid tumor having one or more genetic alterations in each of FGFR1 and FGFR3. In some embodiments, the human patient has a solid tumor having one or more genetic alterations in each of FGFR2 and FGFR3. In some embodiments, the human patient has a solid tumor having one or more genetic alterations in each of FGFR1, FGFR2, and FGFR3. Additionally, the human patient may have a solid tumor having multiple genetic alterations in only one of FGFR1, FGFR2, or FGFR3. For example, the human patient may have a solid tumor having one or more amplifications in FGFR1, as well as a M640I mutation. In another non-limiting example, the human patient may have a solid tumor having a Y375C mutation in FGFR3 and a FGFR3-TACC3 fusion.

The one or more genetic alterations of FGFR1, FGFR2, and/or FGFR3 can be determined using any method known in the art. For example, a genetic alteration of FGFR1, FGFR2, and/or FGFR3 can be assessed by fluorescence in situ hybridization (FISH) or next generation sequencing (NGS).

In some embodiments, Compound A is in free base form.

In some embodiments, Compound A is a pharmaceutically acceptable salt. In some embodiments, a pharmaceutically acceptable salt is obtained by reacting Compound A with an acid. In other embodiments, a pharmaceutically acceptable salt is obtained by reacting compound A with a base. In certain embodiments, the pharmaceutically acceptable salt is a malate salt. In certain embodiments, the pharmaceutically acceptable salt is a hydrochloride salt. In certain embodiments, the pharmaceutically acceptable salt is a methane sulfonate salt, such as a methane sulfonate monohydrate salt. In certain alternative embodiments, the pharmaceutically acceptable salt is an acetate salt, a succinate salt, a fumarate salt, a maleate salt, a tartrate salt, a citrate salt, a lactate salt, a stearate salt, a benzoate salt or a p-toluenesulfonate salt.

In some embodiments, the amount of Compound A, or pharmaceutically acceptable salt thereof, that is administered corresponds to an amount between about 40 mg and about 150 mg of Compound A, as its free base, daily. In certain embodiments, the amount of Compound A, or pharmaceutically acceptable salt thereof, corresponds to an amount of about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg of Compound A, as its free base, daily. In certain embodiments, the amount of Compound A, or pharmaceutically acceptable salt thereof, corresponds to an amount of about 60 mg, about 70 mg, about 80 mg, about 90 mg, about 100 mg, about 110 mg, about 120 mg, about 130 mg, about 140 mg, or about 150 mg of Compound A, as its free base, daily. In certain embodiments, the amount of Compound A, or pharmaceutically acceptable salt thereof, corresponds to an amount of about 80 mg of Compound A, as its free base, daily.

The amount of Compound A to be administered, as a pharmaceutically acceptable salt, will be adjusted based on the weight of the salt form. For example, in some embodiments, the amount of Compound A that is administered corresponds to an amount between about 55 mg and about 206 mg of a malate salt of Compound A, daily. In certain embodiments, the amount of Compound A corresponds to an amount of about 55 mg, about 62 mg, about 69 mg, about 76 mg, about 83 mg, about 89 mg, about 96 mg, about 103 mg, about 110 mg, about 117 mg, about 124 mg, about 131 mg, about 138 mg, about 144 mg, about 151 mg, about 158 mg, about 165 mg, about 172 mg, about 179 mg, about 186 mg, about 193 mg, about 199 mg, or about 206 mg of a malate salt of Compound A, daily. In certain embodiments, the daily amount of Compound A corresponds to about 83 mg, about 96 mg, about 110 mg, about 124 mg, about 138 mg, about 151 mg, about 165 mg, about 179 mg, about 193 mg, or about 206 mg of a malate salt of Compound A. In some embodiments, the daily amount of Compound A corresponds to about 110 mg of a malate salt of Compound A.

In some embodiments, Compound A, or pharmaceutically acceptable salt thereof, is administered orally. In certain embodiments, Compound A, or pharmaceutically acceptable salt thereof, is administered in tablet or capsule form. In certain embodiments, Compound A, or pharmaceutically acceptable salt thereof, is administered in tablet form.

In certain embodiments, the tablet or capsule comprises an amount of Compound A, or pharmaceutically acceptable salt thereof, that corresponds to an amount between about 10 mg and about 150 mg of Compound A, as its free base. In certain embodiments, the tablet or capsule comprises an amount of Compound A, or pharmaceutically acceptable salt thereof, that corresponds to about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg of Compound A, as its free base. In certain embodiments, the tablet or capsule comprises an amount of Compound A, or pharmaceutically acceptable salt thereof, corresponding to about 20 mg, about 30 mg, about 50 mg, about 60 mg, about 80 mg, or about 100 mg of Compound A as its free base. In certain embodiments, the tablet or capsule comprises an amount of Compound A, or pharmaceutically acceptable salt thereof, corresponding to about 20 mg of Compound A as its free base. In certain embodiments, the tablet or capsule comprises an amount of Compound A, or pharmaceutically acceptable salt thereof, corresponding to about 30 mg of Compound A as its free base. In certain embodiments, the tablet or capsule comprises an amount of Compound A, or pharmaceutically acceptable salt thereof, corresponding to about 50 mg of Compound A as its free base. In certain embodiments, the tablet or capsule comprises an amount of Compound A, or pharmaceutically acceptable salt thereof, corresponding to about 80 mg of Compound A as its free base.

The amount of Compound A, as a pharmaceutically acceptable salt, will be adjusted based on the weight of the salt form to be included in the tablet or capsule. For example, in some embodiments, the tablet or capsule comprises an amount between about 14 mg and about 206 mg of a malate salt of Compound A. In certain embodiments, the tablet or capsule comprises an amount of about 14 mg, about 21 mg, about 28 mg, about 34 mg, about 41 mg, about 48 mg, about 55 mg, about 62 mg, about 69 mg, about 76 mg, about 83 mg, about 89 mg, about 96 mg, about 103 mg, about 110 mg, about 117 mg, about 124 mg, about 131 mg, about 138 mg, about 144 mg, about 151 mg, about 158 mg, about 165 mg, about 172 mg, about 179 mg, about 186 mg, about 193 mg, about 199 mg, or about 206 mg of a malate salt of Compound A. In certain embodiments, the tablet or capsule comprises an amount of about 28 mg, about 41 mg, about 69 mg, about 83 mg, about 110 mg or about 138 mg of a malate salt of Compound A. In some embodiments, the tablet or capsule comprises about 110 mg of a malate salt of Compound A.

In some embodiments, the daily dose of Compound A, or pharmaceutically acceptable salt thereof, is administered as one dose one time per day. In certain embodiments, the daily dose of Compound A, or pharmaceutically acceptable salt thereof, is divided into multiple doses that are administered one, two, three, or four times per day. For example, about 80 mg of Compound A as its free base, administered as its malate salt, could be administered as (1) a single tablet comprising an amount of Compound A, as a malate salt, corresponding to about 80 mg of Compound A, as its free base, once daily, (2) four tablets comprising an amount of Compound A, as a malate salt, corresponding to about 20 mg of Compound A, as its free base, in the morning, or (3) two tablets comprising an amount of Compound A, as a malate salt, corresponding to about 20 mg of Compound A, as its free base, in the morning and two tablets comprising an amount of Compound A, as a malate salt, corresponding to about 20 mg of Compound A, as its free base, in the evening. For example, about 110 mg of a malate salt of Compound A could be administered as (1) a single tablet comprising an amount of about 110 mg of a malate salt of Compound A, once daily, (2) four tablets comprising an amount about 28 mg of a malate salt of Compound A, in the morning, or (3) two tablets comprising an amount about 28 mg of a malate salt of Compound A, in the morning and two tablets comprising an amount about 28 mg of a malate salt of Compound A, in the evening.

In some embodiments, the methods involve administering Compound A, or pharmaceutically acceptable salt thereof, in an amount corresponding to an amount of between about 40 mg and about 150 mg of Compound A, as its free base, daily to a human patient having a solid tumor. In some embodiments, the human patient has a solid tumor selected from the group consisting of biliary tract cancer, breast cancer, cholangiocarcinoma, urothelial cancer, uterine neoplasm, lung adenocarcinoma, squamous non-small cell lung cancer, non-squamous non-small cell lung cancer, gastric cancer, sarcoma, bladder cancer, head and neck cancer, small cell lung cancer, endometrial cancer, esophageal cancer, adenoid cystic carcinoma, gallbladder cancer, colon cancer, thyroid cancer, hepatocellular cancer, prostate cancer, oral cancer, cervical cancer, pancreatic carcinoma, ovarian cancer, and serous carcinoma to the peritoneum. In some embodiments, the cholangiocarcinoma is selected from the group consisting of intrahepatic cholangiocarcinoma, perihilar cholangiocarcinoma, and distal cholangiocarcinoma.

In some embodiments, the human patient previously received at least one prior standard therapy. In certain embodiments, the prior standard therapy comprises chemotherapy. In certain embodiments, the prior therapy comprises administering gemcitabine. In certain embodiments, the prior standard therapy comprises an anti-PD1/PDL1 therapy. In certain embodiments, the prior standard therapy comprises a combination of chemotherapy and an anti-PD1/PDL1 therapy. In certain embodiments, the prior standard therapy comprises radiotherapy. In certain embodiments, the prior standard therapy comprises a combination of radiotherapy and chemotherapy.

In some embodiments, the human patient has relapsed or progressed after administration of at least one prior standard therapy. In certain embodiments, the human patient has biliary tract cancer and has relapsed or progressed after administration of at least one prior standard therapy. In certain embodiments, the human patient has biliary tract cancer and has relapsed or progressed after administration of chemotherapy (e.g., gemcitabine). In certain embodiments, the human patient has urothelial cancer and has relapsed or progressed after administration of at least one prior standard therapy. In certain embodiments, the human patient has urothelial cancer and has relapsed or progressed after administration of chemotherapy, an anti-PD1/PDL1 therapy, or a combination of chemotherapy and an anti-PD1/PDL1 therapy. In some embodiments, the chemotherapy is cisplatin-based or carboplatin-based chemotherapy. In some embodiments, the human patient has non-small cell lung cancer and has relapsed or progressed after administration at least one prior standard therapy. In some embodiments, the human patient has non-small cell lung cancer and has relapsed or progressed after administration of chemotherapy, an anti-PD1/PDL1 therapy, or a combination of chemotherapy and an anti-PD1/PDL1 therapy.

In some embodiments, Compound A, or pharmaceutically acceptable salt thereof, is administered to the human patient after the patient has fasted for four hours. In some embodiments, the human patient fasts for two hours after Compound A, or pharmaceutically acceptable salt thereof, is administered to the human patient.

### EXAMPLES

It is understood that the examples and embodiments described herein are for illustrative purposes only and that various modifications or changes in light thereof will be suggested to persons skilled in the art and are to be included within the spirit and purview of this application.

### Example 1: Debio 1347 Dose Escalation in Advanced Solid Tumors

The dose escalation part of a phase I, gene alteration-based, open label, multicenter study of oral Debio 1347 in patients with advanced solid malignancies, whose tumors have an alteration of the FGFR 1, 2 or 3 genes, was conducted (Debio 1347-101).

Patients with advanced solid tumors who failed to respond to standard treatment (and had received no prior FGFR treatment), harboring FGFR 1, 2 or 3 gene alterations received escalating doses of Debio 1347 (Compound A, in its malate salt form) starting from 10 mg (based on the amount of the free base) once daily. Dose escalation followed a 3+3+3 algorithm based on a modified Fibonacci sequence. The maximum tolerated dose (MTD) was defined as the highest dose level at which 2 of 3, 3 of 6 or 3 of 9 patients experienced a dose limiting toxicity (DLT) during the first 28 days of treatment. Tumor response was assessed as secondary endpoint according to RECIST (version 1.1) at 6 weeks from baseline, at Cycle 4 and every 3 courses from Cycle 6. An independent radiological review of patients who responded to treatment and a “post hoc” analysis to confirm the FGFR genetic alterations were performed centrally. Pharmacokinetics (PK) and pharmacodynamics were serially evaluated in blood, skin and/or tumor tissue.

Fifty-eight patients were treated, including patients with mutations (n=19), amplifications (n=31) and fusions (n=12). The dose was escalated up to 150 mg (based on the amount of the free base) over 8 cohorts. One patient had high-level amplification in FGFR2 with an activating mutation on the amplicon. As shown in Table 1 below, overall, FGFR1 gene amplification was predominant; FGFR2 and FGFR3 genes showed mutations as the most frequent type of alteration.

TABLE 1
Patient baseline characteristics (n = 58)

| | | |
|---|---|---|
| Age, years | Median ± SD (range) | 58.5 ± 11.4 (26-79) |
| Sex | Female | 36 (62.1%) |
| | Male | 22 (37.9%) |
| Race | White | 51 (87.9%) |
| | Black or African American | 2 (3.4%) |
| | Asian | 2 (3.4%) |
| | Other | 3 (5.2%) |
| ECOG | 0 | 20 (34.5%) |
| | 1 | 37 (67.8%) |
| | 2 | 1 (1.7%) |
| Tumor type | Breast cancer | 12 (20.7%) |
| | Cholangiocarcinoma | 6 (10.3%) |
| | Urothelial carcinoma | 6 (10.3%) |
| | Uterine neoplasm | 5 (8.6%) |
| | sqNSCLC | 4 (6.9%) |
| | Gastric cancer | 4 (6.9%) |
| | Prostate cancer | 2 (3.4%) |
| | Gallbladder | 2 (3.4%) |
| | Cervical cancer | 2 (3.4%) |
| | Others (each with only one | 15 (25.9%) |
| | occurrence) | |

FGFR gene alteration status

| | | |
|---|---|---|
| FGFR1 | Amplification | 23 (39.7%) |
| | Fusion | 1 (1.7%) |
| | Mutation | 2 (3.4%) |
| FGFR2 | Amplification | 5 (8.6%) |
| | Fusion | 6 (10.3%) |
| | Mutation | 7 (12.1%) |
| FGFR3 | Amplification | 4 (6.9%) |
| | Fusion | 5 (8.6%) |
| | Mutation | 10 (17.2%) |

Noteworthy, some patients also displayed combinations of amplifications with fusions/translocations or mutations. The post-hoc analysis did not confirm local FGFR results in 9 patients (4 amplifications; 3 mutations; and 2 fusions). For 15 patients, no post-hoc analysis was performed due to missing archival biopsy or poor quality of the remaining biopsy material.

All 58 treated patients had ≥1 treatment emergent adverse event (TEAE), of which the highest severity was of grade 1 in 4 (6.9%), grade 2 in 17 (29.3%), grade 3 in 28 (48.3%), grade 4 in 2 (3.4%) and grade 5 in 7 (12.1%) patients. Overall, 21 patients died from disease progression; no death was considered drug-related. The dosages listed below are the amounts corresponding to the free base form of Debio 1347 (administered as a malate salt).

The most common TEAEs with an incidence ≥25% were hyperphosphatemia, diarrhea, nausea, fatigue, constipation, decreased appetite, nail changes, and dry mouth. Most often severe (i.e. of grade >2) were hyperphosphatemia, anemia, hyponatremia, and dyspnea. In total, 20 patients experienced 39 serious adverse effects (SAEs), most commonly dyspnea, but only two SAEs were deemed possibly study drug related and thus DLTs, i.e. hyperamylasemia (80 mg) and stomatitis (110 mg). DLTs occurred in 3 other patients, i.e. grade-2 dry mouth and eyes (60 mg); grade-3 asymptomatic hypercalcemia (80 mg); and grade-3 bilirubin increase and hyperphosphatemia (110 mg). The patient with hyperamylasemia died from disease progression. The remaining 4 patients recovered after Debio 1347 interruption and/or dose modification. As there were only ≤2 DLTs at the same dose level, the MTD was not formally reached. Dose modifications due to adverse events were required by 30 (51.7%) patients. At ≥110 mg, 7 out of 9 patients required a dose reduction after completion of the 28-day DLT period, predominantly due to hyperphosphatemia, stomatitis, skin, and nail toxicity. At 60 and 80 mg, these were only required in 20% of patients and up to 40 mg, no dose reduction was required at all.

Pharmacokinetics appeared overall linear, with a mean half-life of 11.5 hours; hyperphosphatemia was dose-dependent. The patients exhibited the following tumor types: 20.7% breast cancer (n=12), 10.3% cholangiocarcinoma (n=6), 10.3% urothelial cancer (n=6), 8.6% uterine neoplasm (n=5), 6.9% sqNSCLC (n=4), 6.9% gastric cancer (n=4), 3.4% cervical cancer (n=2), 3.4% prostate cancer (n=2), 3.4% gallbladder cancer (n=2), and 25.9% others (n=15).

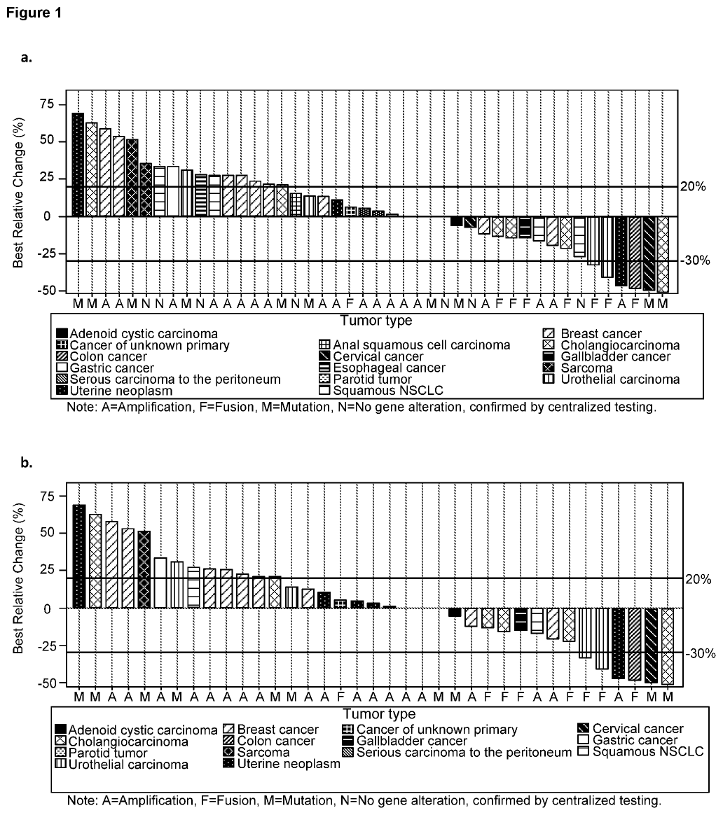

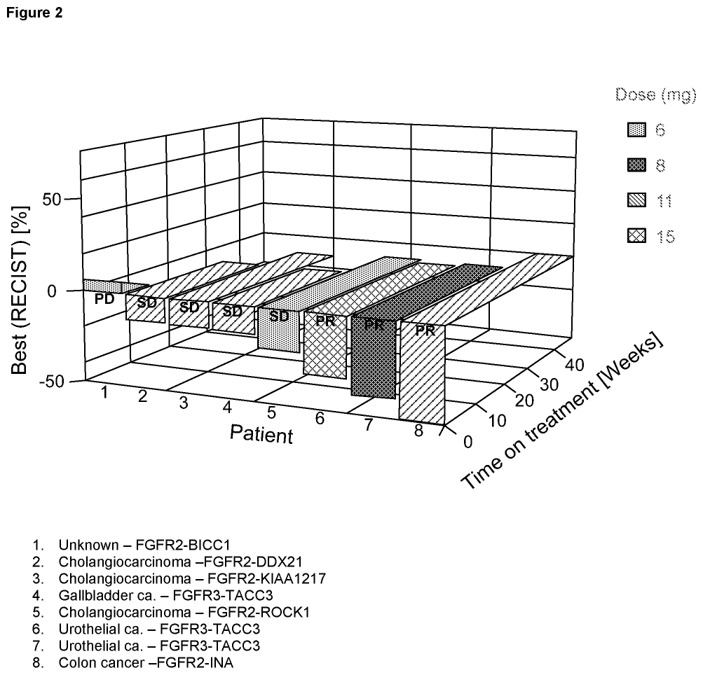

A total of 57 patients were evaluable for tumor response. One patient had no evaluable disease, i.e. neither clinical assessment nor adequate radiographic assessment of tumor response. Overall, 6 patients had PRs (FIG. 1(a) and FIG. 1(b)). Of these six PRs, two had urothelial carcinoma (FGFR3 fusion—80, 150 mg), the others uterine (FGFR1 amplified—30 mg), cervical (FGFR2 amplified—80 mg), colon cancer (FGFR1 amplified—110 mg) or cholangiocarcinoma (FGFR2 mutation—110 mg). For the latter the independent centralized radiological review changed response from partial to confirmed completed response (CR). Among 10 patients with confirmed FGFR fusions treated at doses ≥60 mg/day, 3 achieved PRs and 5 disease stabilization (FIG. 2). This represents a disease control rate (DCR) in this subpopulation of 80% (versus 38.6% overall). The median treatment duration in patients with disease control was 34 weeks (range: 24-47). Among the remaining patients, 35 patients had progressive disease, 13 per clinical assessment in absence of radiographic evidence; 16 patients had stable disease, of whom 10 showed reduced target lesion size ≤30% at least once on-treatment.

Specifically, patients having the following genetic alterations in FGFR1, 2, or 3 were administered Debio 1347: FGFR1 amplification, FGFR2 amplification, FGFR3 amplification, M640I mutation in FGFR2, G384R mutation in FGFR2, T394I mutation in FGFR2, N549K mutation in FGFR2, Y805fs11 mutation in FGFR2, S529C mutation in FGFR2, Y375C mutation in FGFR3, S249C mutation in FGFR3, G561A mutation in FGFR3, V684L mutation in FGFR3, T689M mutation in FGFR3, R248C mutation in FGFR3, D758N mutation in FGFR3, FGFR2-DDX21 fusion, FGFR2-KIAA1217 fusion, FGFR2-ROCK1 fusion, FGFR2-BICC1 fusion, FGFR2-INA fusion, FGFR2-CD44 fusion, FGFR3-TACC3 fusion.

Additionally, one patient with intrahepatic cholangiocarcinoma patient having an FGFR2 deletion of exon 5 exhibited a partial response lasting up to 48 weeks was observed in an (FGFR2 deletion of exon 5). This partial response was later requalified as a complete response upon independent radiological review.

Debio 1347 had a tolerable and manageable safety profile. Encouraging antitumor activity was seen in several tumor types, mainly in patients with FGFR2 or 3 gene alterations, including fusion/translocation events, treated at 80 mg and 110 mg daily. The dose escalation part of the Debio 1347-101 phase I study allowed to establish a recommended phase II dose (RP2D) of Debio 1347 at 80 mg daily.

### Example 2: Debio 1347 Expansion Part in Advanced Solid Tumors

The expansion part of the Debio 1347-101 phase I study includes a cohort of patients with advanced solid malignancies of any histological tumor type harboring an FGFR 1, 2 or 3 fusion and for whom standard treatment does not exist or is not indicated. The primary objective of this expansion phase is to confirm the safety profile at the RP2D of 80 mg once daily.

As of January 2019, two patients achieved a BOR of PR by investigator assessment according to RECIST 1.1 criteria, one suffering from a cholangiocarcinoma harboring a FGFR2-TACC2 fusion and one suffering from a colon cancer harboring a FGFR2-RP11-89K10.1 fusion. Another patient suffering from a cholangiocarcinoma FGFR2-ATAD2 fusion achieved a disease stabilization (SD) with tumor volume reduction of about 7% after 6 weeks of treatment.

### Example 3: Debio 1347 Phase II Basket Trial

A multicentre, basket, two stage, adaptive single arm Phase II study in subjects with solid tumors harboring FGFR1-3 gene fusion/rearrangement will be conducted. Three cohorts will be included consisting of subjects with biliary tract cancer (Cohort 1), urothelial cancer (Cohort 2) and all other solid tumor histologies not included in Cohorts 1-2 such as NSCLC, head and neck cancer, thyroid cancer, oral cancer, breast cancer, prostate cancer and others but excluding primary brain tumors (Cohort 3).

Subjects with locally advanced (unresectable) or metastatic tumors with FGFR1-3 gene fusion/rearrangement who require systemic therapy and who have radiologic and/or clinical progression following at least one prior standard treatment or who have no satisfactory alternative treatment options will be included in this study, according to inclusion/exclusion criteria specifically defined per protocol.

Enrolled subjects will receive Debio 1347 tablets orally at the dose of 80 mg once daily from Day 1 to Day 28 in 28-day cycles until occurrence of any end-of-treatment or end-of-study criteria occurs, such as disease progression, unacceptable toxicity or withdrawal of consent.

Individual dose adjustment of Debio 1347 may be considered and applied as per protocol if required. The primary endpoint of this study is the objective response rate (ORR), defined as the proportion of subjects with a best overall response (BOR) of partial or complete response, as centrally measured by RECIST 1.1 criteria.

The secondary endpoints of this study are:

Duration of response (DoR), defined as the time from the date of the initial response to date of the first documented progression or death due to any cause.

Disease control rate (DCR), defined as the proportion of subjects with a BOR of CR or PR or SD.

Progression-free survival (PFS), defined as the time from the start date of treatment to date of the first documented progression or death due to any cause.

Overall survival (OS), defined as the time from the start date of treatment to date of death due to any cause.

Proportion of subjects with TEAEs assessed by NCI-CTCAE v5.0 (published Nov. 27, 2017 by the U.S. Department of Health and Human Services) and SAEs.

Debio 1347 plasma exposure and relationships with efficacy and safety endpoints (including notably changes in electrocardiogram QT interval).

It is to be appreciated that the Detailed Description section, and not the Summary and Abstract sections, is intended to be used to interpret the claims. The Summary and Abstract sections sets forth one or more, but not all, exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

What is claimed:

1. A method for treating a human patient having a solid tumor comprising administering to the patient, in need thereof, Compound A, or pharmaceutically acceptable salt thereof, in an amount corresponding to an amount between about 40 mg and about 150 mg of Compound A, as its free base, daily.

2. The method of claim 1, wherein the human patient has a solid tumor with one or more genetic alterations in FGFR1, FGFR2, and/or FGFR3.

3. The method of claim 2, wherein the human patient has a solid tumor with one or more genetic alterations in FGFR1, FGFR2, and/or FGFR3 that is an amplification, an activating mutation, a deletion, and/or a fusion/translocation.

4. The method of any one of claims 1-3, wherein the human patient has a solid tumor with one or more genetic alterations that is one or more fusions/translocations in FGFR1, FGFR2, and/or FGFR3.

5. The method of any one of claims 1-4, wherein the human patient has a solid tumor having a FGFR1 fusion/translocation.

6. The method of any one of claims 1-5, wherein the human patient has a solid tumor having a FGFR2 fusion/translocation.

7. The method of claim 6, wherein the FGFR2 fusion/translocation is selected from the group consisting of FGFR2-DDX21, FGFR2-KIAA1217, FGFR2-ROCK1, FGFR2-BICC1, FGFR2-INA, FGFR2-CD44, FGFR2-TACC2, FGFR2-RP11-89K10.1, and FGFR2-ATAD2.

8. The method of any one of claims 1-7, wherein the human patient has a solid tumor having a FGFR3 fusion/translocation.

9. The method of claim 8, wherein the FGFR3 fusion/translocation is FGFR3-TACC3.

10. The method of any one of claims 1-9, wherein the human patient has a solid tumor with one or more genetic alterations that is one or more mutations in FGFR1, FGFR2, and/or FGFR3.

11. The method of any one of claims 1-10, wherein the human patient has a solid tumor with a genetic alteration that is a mutation in FGFR1.

12. The method of any one of claims 1-11, wherein the human patient has a solid tumor with a genetic alteration that is a mutation in FGFR2.

13. The method of claim 12, wherein the mutation in FGFR2 is one or more selected from the group consisting of M640I, G384R, T394I, N549K, Y805fs11, and S529C.

14. The method of any one of claims 1-13, wherein the human patient has a solid tumor with a genetic alteration that is a mutation in FGFR3.

15. The method of claim 14, wherein the mutation in FGFR3 is one or more selected from the group consisting of Y375C, S249C, G561A, V684L, T689M, R248C, and D758N.

16. The method of any one of claims 1-15, wherein the human patient has a solid tumor with one or more genetic alterations that is one or more amplifications in FGFR1, FGFR2, and/or FGFR3.

17. The method of any one of claims 1-16, wherein the human patient has a solid tumor with a genetic alteration that is an amplification in FGFR1.

18. The method of any one of claims 1-17, wherein the human patient has a solid tumor with a genetic alteration that is an amplification in FGFR2.

19. The method of any one of claims 1-18, wherein the human patient has a solid tumor with a genetic alteration that is an amplification in FGFR3.

20. The method of any one of claims 1-19, wherein the human patient has a solid tumor with one or more genetic alterations that is one or more deletions in FGFR1, FGFR2, and/or FGFR3.

21. The method of any one of claims 1-20, wherein the human patient has a solid tumor with a genetic alteration that is a deletion in FGFR1.

22. The method of any one of claims 1-21, wherein the human patient has a solid tumor with a genetic alteration that is a deletion in FGFR2.

23. The method of any one of claims 1-22, wherein the human patient has a solid tumor with a deletion in exon 5 of FGFR2.

24. The method of any one of claims 1-23, wherein the human patient has a solid tumor with a deletion that is H167-N173del in exon 5 of FGFR2.

25. The method of any one of claims 1-24, wherein the human patient has a solid tumor with a genetic alteration that is a deletion in FGFR3.

26. The method of any one of claims 1-25, wherein Compound A is in free base form.

27. The method of any one of claims 1-25, wherein Compound A is a pharmaceutically acceptable salt.

28. The method of claim 27, wherein the pharmaceutically acceptable salt is a malate salt, a hydrochloride salt, a methane sulfonate salt, an acetate salt, a succinate salt, a fumarate salt, a maleate salt, a tartrate salt, a citrate salt, a lactate salt, a stearate salt, a benzoate salt or a p-toluenesulfonate salt.

29. The method of claim 28, wherein the pharmaceutically acceptable salt is a malate salt.

30. The method of any one of claims 1-29, wherein the human patient has a solid tumor selected from the group consisting of biliary tract cancer, breast cancer, cholangiocarcinoma, urothelial cancer, uterine neoplasm, lung adenocarcinoma, squamous non-small cell lung cancer, non-squamous non-small cell lung cancer, gastric cancer, sarcoma, bladder cancer, head and neck cancer, small cell lung cancer, endometrial cancer, esophageal cancer, adenoid cystic carcinoma, gallbladder cancer, colon cancer, thyroid cancer, hepatocellular cancer, prostate cancer, oral cancer, cervical cancer, pancreatic carcinoma, ovarian cancer, and serous carcinoma to the peritoneum.

31. The method of claim 30, wherein the cholangiocarcinoma is selected from the group consisting of intrahepatic cholangiocarcinoma, perihilar cholangiocarcinoma, and distal cholangiocarcinoma.

32. The method of any one of claims 1-31, wherein the amount of Compound A, or pharmaceutically acceptable salt thereof, is an amount corresponding to about 60 mg, about 70 mg, about 80 mg, about 90 mg, about 100 mg, about 110 mg, about 120 mg, about 130 mg, about 140 mg, or about 150 mg of Compound A, as its free base, daily.

33. The method of claim 32, wherein the amount of Compound A, or pharmaceutically acceptable salt thereof, is an amount corresponding to about 80 mg of Compound A, as its free base, daily.

34. The method of any one of claims 1-31, wherein Compound A is a malate salt in an amount of about 83 mg, about 96 mg, about 110 mg, about 124 mg, about 138 mg, about 151 mg, about 165 mg, about 179 mg, about 193 mg, or about 206 mg daily.

35. The method of claim 34, wherein the amount of Compound A as a malate salt is about 110 mg daily.

36. The method of any one of claims 1-35, wherein Compound A, or pharmaceutically acceptable salt thereof, is administered orally.

37. The method of any one of claims 1-36, wherein Compound A, or pharmaceutically acceptable salt thereof, is administered in capsule form.

38. The method of any one of claims 1-36, wherein Compound A, or pharmaceutically acceptable salt thereof, is administered in tablet form.

39. The method of claim 38, wherein the tablet comprises an amount of Compound A, or pharmaceutically acceptable salt thereof, corresponding to an amount of about 20 mg, about 30 mg, about 50 mg, about 60 mg, about 80 mg or about 100 mg of Compound A, as its free base.

40. The method of claim 39, wherein the tablet comprises an amount of Compound A, or pharmaceutically acceptable salt thereof, corresponding to an amount of about 80 mg of Compound A, as its free base.

41. The method of claim 38, wherein the tablet comprises an amount of Compound A as its malate salt of about 28 mg, about 41 mg, about 69 mg, about 83 mg, about 110 mg or about 138 mg.

42. The method of claim 41, wherein the tablet comprises an amount of Compound A as its malate salt of about 110 mg.

43. The method of any one of claims 1-42, wherein Compound A, or pharmaceutically acceptable salt thereof, is administered as one dose one time per day.

44. The method of any one of claims 1-43, wherein Compound A, or pharmaceutically acceptable salt thereof, is divided into multiple doses that are administered one, two, three, or four times per day.

45. The method of any one of claims 1-44, wherein Compound A, or pharmaceutically acceptable salt thereof, is administered to the human patient after the patient has fasted for four hours.

46. The method of any one of claims 1-45, wherein the human patient fasts for two hours after Compound A, or pharmaceutically acceptable salt thereof, is administered to the human patient.

47. The method of any one of claims 1-46, wherein the human patient has relapsed or progressed after administration of at least one prior standard therapy.

48. The method of claim 47, wherein the human patient has biliary tract cancer.

49. The method of claim 48, wherein the human patient has relapsed or progressed after administration of gemcitabine-based chemotherapy.

50. The method of claim 47, wherein the human patient has urothelial cancer.

51. The method of claim 50, wherein the human patient has relapsed or progressed after administration of chemotherapy, an anti-PD1/PDL1 therapy, or a combination of chemotherapy and an anti-PD1/PDL1 therapy.

52. The method of claim 51, wherein the chemotherapy is cisplatin-based or carboplatin-based chemotherapy.

53. The method of claim 47, wherein the human patient has non-small cell lung cancer.

54. The method of claim 53, wherein the human patient has relapsed or progressed after administration of chemotherapy, an anti-PD1/PDL1 therapy, or a combination of prior chemotherapy and an anti-PD1/PDL1 therapy.

55. A method of treating a human patient having a solid tumor with one or more gene fusions/translocations in FGFR1, FGFR2, and/or FGFR3 comprising orally administering an amount of Compound A, or pharmaceutically acceptable salt thereof, in amount corresponding to about 80 mg of Compound A, as its free base, daily.

56. The method of claim 55, wherein the human patient has a solid tumor having a FGFR1 fusion.

57. The method of claim 55 or 56, wherein the human patient has a solid tumor having a FGFR2 fusion.

58. The method of claim 57, wherein the FGFR2 fusion is selected from the group consisting of FGFR2-DDX21, FGFR2-KIAA1217, FGFR2-ROCK1, FGFR2-BICC1, FGFR2-INA, FGFR2-CD44, FGFR2-TACC2, FGFR2-RP11-89K10.1, and FGFR2-ATAD2.

59. The method of any one of claims 55-58, wherein the human patient has a solid tumor having a FGFR3 fusion.

60. The method of claim 59, wherein the FGFR3 fusion is FGFR3-TACC3.

61. The method of any one of claims 55-60, wherein the human patient has a solid tumor having a deletion in FGFR2.

62. The method of claim 61, wherein the deletion in FGFR2 is in exon 5.

63. The method of claim 62, wherein the deletion is H167-N173del.

64. The method of any one of claims 55-63, wherein the human patient has a solid tumor with an additional one or more genetic alterations that is one or more mutations in FGFR1, FGFR2, and/or FGFR3.

65. The method of claim 64, wherein the human patient has a solid tumor with an additional genetic alteration that is a mutation in FGFR1.

66. The method of any one of claims 55-65, wherein the human patient has a solid tumor with an additional genetic alteration that is a mutation in FGFR2.

67. The method of claim 66, wherein the mutation in FGFR2 is one or more selected from the group consisting of M640I, G384R, T394I, N549K, Y805fs11, and S529C.

68. The method of any one of claims 55-67, wherein the human patient has a solid tumor with an additional genetic alteration that is a mutation in FGFR3.

69. The method of claim 68, wherein the mutation in FGFR3 is one or more selected from the group consisting of Y375C, S249C, G561A, V684L, T689M, R248C, and D758N.

70. The method of any one of claims 55-69, wherein the human patient has a solid tumor with an additional one or more genetic alterations that is one or more amplifications in FGFR1, FGFR2, and/or FGFR3.

71. The method of any one of claims 55-70, wherein the human patient has a solid tumor with an additional genetic alteration that is an amplification in FGFR1.

72. The method of any one of claims 55-71, wherein the human patient has a solid tumor with an additional genetic alteration that is an amplification in FGFR2.

73. The method of any one of claims 55-72, wherein the human patient has a solid tumor with an additional genetic alteration that is an amplification in FGFR3.

74. The method of any one of claims 55-73, wherein Compound A is in free base form.

75. The method of any one of claims 55-73, wherein Compound A is a pharmaceutically acceptable salt.

76. The method of claim 75, wherein the pharmaceutically acceptable salt is a malate salt, a hydrochloride salt, a methane sulfonate salt, an acetate salt, a succinate salt, a fumarate salt, a maleate salt, a tartrate salt, a citrate salt, a lactate salt, a stearate salt, a benzoate salt or a p-toluenesulfonate salt.

77. The method of claim 76, wherein the pharmaceutically acceptable salt is a malate salt.

78. The method of any one of claims 55-77, wherein the human patient has a solid tumor selected from the group consisting of biliary tract cancer, breast cancer, cholangiocarcinoma, urothelial cancer, uterine neoplasm, lung adenocarcinoma, squamous non-small cell lung cancer, non-squamous non-small cell lung cancer, gastric cancer, sarcoma, bladder cancer, head and neck cancer, small cell lung cancer, endometrial cancer, esophageal cancer, adenoid cystic carcinoma, gallbladder cancer, colon cancer, thyroid cancer, hepatocellular cancer, prostate cancer, oral cancer, cervical cancer, pancreatic carcinoma, ovarian cancer, and serous carcinoma to the peritoneum.

79. The method of claim 78, wherein the cholangiocarcinoma is selected from the group consisting of intrahepatic cholangiocarcinoma, perihilar cholangiocarcinoma, and distal cholangiocarcinoma.

80. The method of any one of claims 55-79, wherein Compound A, or pharmaceutically acceptable salt thereof, is administered in capsule form.

81. The method of any one of claims 55-79, wherein Compound A, or pharmaceutically acceptable salt thereof, is administered in tablet form.

82. The method of claim 81, wherein the tablet comprises an amount of Compound A, or pharmaceutically acceptable salt thereof, corresponding to an amount of about 20 mg, or about 80 mg of Compound A, as its free base.

83. The method of claim 82, wherein the tablet comprises an amount of Compound A, or pharmaceutically acceptable salt thereof, corresponding to an amount of about 80 mg of Compound A, as its free base.

84. The method of any one of claims 55-83, wherein the tablet comprises an amount of Compound A is a malate salt in an amount of about 28 mg or about 110 mg daily.

85. The method of claim 84, wherein the tablet comprises an amount of Compound A as a malate salt is about 110 mg daily.

86. The method of any one of claims 55-85, wherein Compound A, or pharmaceutically acceptable salt thereof, is administered as one dose one time per day.

87. The method of any one of claims 55-85, wherein Compound A, or pharmaceutically acceptable salt thereof, is divided into multiple doses that are administered one, two, three, or four times per day.

88. The method of any one of claims 55-87, wherein Compound A, or pharmaceutically acceptable salt thereof is administered to the human patient after the patient has fasted for four hours.

89. The method of any one of claims 55-88, wherein the human patient fasts for two hours after Compound A, or pharmaceutically acceptable salt thereof, is administered to the human patient.

90. The method of any one of claims 55-89, wherein the human patient has relapsed or progressed after administration of at least one prior standard therapy.

91. The method of claim 90, wherein the human patient has biliary tract cancer.

92. The method of claim 91, wherein the human patient has relapsed or progressed after administration of gemcitabine-based chemotherapy.

93. The method of claim 90, wherein the human patient has urothelial cancer.

94. The method of claim 93, wherein the human patient has relapsed or progressed after administration of chemotherapy, an anti-PD1/PDL1 therapy, or a combination of chemotherapy and an anti-PD1/PDL1 therapy.

95. The method of claim 94, wherein chemotherapy is cisplatin-based or carboplatin-based chemotherapy.

96. The method of claim 90, wherein the human patient has non-small cell lung cancer.

97. The method of claim 96, wherein the human patient has relapsed or progressed after administration of chemotherapy, an anti-PD1/PDL1 therapy, or a combination of chemotherapy and an anti-PD1/PDL1 therapy.

98. A method for treating a human patient having a solid tumor with one or more genetic alterations in FGFR1, FGFR2, and/or FGFR3, the method comprising the steps of:
(a) obtaining or having obtained a biological sample from the patient;
(b) performing or having performed an assay on the biological sample to determine if the patient has one or more genetic alterations in FGFR1, FGFR2, and/or FGFR3; and
(c) administering an amount of Compound A, or pharmaceutically acceptable salt thereof, corresponding to an amount between about 40 mg and about 150 mg of Compound A, as its free base, daily.

99. The method of claim 98, wherein the human patient has a solid tumor with one or more genetic alterations in FGFR1, FGFR2, and/or FGFR3 that is an amplification, an activating mutation, and/or a fusion translocation.

100. The method of claim 98 or 99, wherein the human patient has a solid tumor with one or more genetic alterations that is one or more fusions/translocations in FGFR1, FGFR2, and/or FGFR3.

101. The method of any one of claims 98-100, wherein the human patient has a solid tumor having a FGFR1 fusion.

102. The method of any one of claims 98-101, wherein the human patient has a solid tumor having a FGFR2 fusion.

103. The method of claim 102, wherein the FGFR2 fusion is selected from the group consisting of FGFR2-DDX21, FGFR2-KIAA1217, FGFR2-ROCK1, FGFR2-BICC1, FGFR2-INA, FGFR2-CD44, FGFR2-TACC2, FGFR2-RP11-89K10.1, and FGFR2-ATAD2.

104. The method of any one of claims 98-103, wherein the human patient has a solid tumor having a FGFR3 fusion.

105. The method of claim 104, wherein the FGFR3 fusion is FGFR3-TACC3.

106. The method of any one of claims 98-105, wherein the human patient has a solid tumor with one or more genetic alterations that is one or more mutations in FGFR1, FGFR2, and/or FGFR3.

107. The method of any one of claims 98-106, wherein the human patient has a solid tumor with a genetic alteration that is a mutation in FGFR1.

108. The method of any one of claims 98-107, wherein the human patient has a solid tumor with a genetic alteration that is a mutation in FGFR2.

109. The method of claim 108, wherein the mutation in FGFR2 is one or more selected from the group consisting of M640I, G384R, T394I, N549K, Y805fs11, and S529C.

110. The method of any one of claims 98-109, wherein the human patient has a solid tumor with a genetic alteration that is a mutation in FGFR3.

111. The method of claim 110, wherein the mutation in FGFR3 is one or more selected from the group consisting of Y375C, S249C, G561A, V684L, T689M, R248C, and D758N.

112. The method of any one of claims 98-111, wherein the human patient has a solid tumor having a deletion in FGFR2.

113. The method of claim 112, wherein the deletion in FGFR2 is in exon 5.

114. The method of claim 113, wherein the deletion is H167-N173del.

115. The method of any one of claims 98-114, wherein the human patient has a solid tumor with one or more genetic alterations that is one or more amplifications in FGFR1, FGFR2, and/or FGFR3.

116. The method of any one of claims 98-115, wherein the human patient has a solid tumor with a genetic alteration that is an amplification in FGFR1.

117. The method of any one of claims 98-116, wherein the human patient has a solid tumor with a genetic alteration that is an amplification in FGFR2.

118. The method of any one of claims 98-117, wherein the human patient has a solid tumor with a genetic alteration that is an amplification in FGFR3.

119. The method of any one of claims 98-118, wherein Compound A is in free base form.

120. The method of any one of claims 98-118, wherein Compound A is a pharmaceutically acceptable salt.

121. The method of claim 120, wherein the pharmaceutically acceptable salt is a malate salt, a hydrochloride salt, a methane sulfonate salt, an acetate salt, a succinate salt, a fumarate salt, a maleate salt, a tartrate salt, a citrate salt, a lactate salt, a stearate salt, a benzoate salt or a p-toluenesulfonate salt.

122. The method of claim 121, wherein the pharmaceutically acceptable salt is a malate salt.

123. The method of any one of claims 98-122, wherein the human patient has a solid tumor selected from the group consisting of biliary tract cancer, breast cancer, cholangiocarcinoma, urothelial cancer, uterine neoplasm, lung adenocarcinoma, squamous non-small cell lung cancer, non-squamous non-small cell lung cancer, gastric cancer, sarcoma, bladder cancer, head and neck cancer, small cell lung cancer, endometrial cancer, esophageal cancer, adenoid cystic carcinoma, gallbladder cancer, colon cancer, thyroid cancer, hepatocellular cancer, prostate cancer, oral cancer, cervical cancer, pancreatic carcinoma, ovarian cancer, and serous carcinoma to the peritoneum.

124. The method of claim 123, wherein the cholangiocarcinoma is selected from the group consisting of intrahepatic cholangiocarcinoma, perihilar cholangiocarcinoma, and distal cholangiocarcinoma.

125. The method of any one of claims 98-124, wherein the amount of Compound A, or its pharmaceutically acceptable salt thereof, corresponds to an amount of about 60 mg, about 70 mg, about 80 mg, about 90 mg, about 100 mg, about 110 mg, about 120 mg, about 130 mg, about 140 mg, or about 150 mg of Compound A, as its free base, daily.

126. The method of claim 125, wherein the amount of Compound A, or pharmaceutically acceptable salt thereof, corresponds to an amount of about 80 mg of Compound A, as its free base, daily.

127. The method of any one of claims 98-124, wherein Compound A is a malate salt in an amount of about 83 mg, about 96 mg, about 110 mg, about 124 mg, about 138 mg, about 151 mg, about 165 mg, about 179 mg, about 193 mg, or about 206 mg daily.

128. The method of claim 127, wherein the amount of Compound A as a malate salt is about 110 mg daily.

129. The method of any one of claims 98-128, wherein Compound A, or its pharmaceutically acceptable salt thereof is administered orally.

130. The method of any one of claims 98-129, wherein Compound A, or pharmaceutically acceptable salt thereof, is administered in capsule form.

131. The method of any one of claims 98-129, wherein Compound A, or pharmaceutically acceptable salt thereof, is administered in tablet form.

132. The method of claim 131, wherein the tablet comprises an amount of Compound A, or pharmaceutically acceptable salt thereof, corresponding to an amount of about 20 mg, about 30 mg, about 50 mg, about 60 mg, about 80 mg, or about 100 mg of Compound A, as its free base.

133. The method of claim 132, wherein the tablet comprises an amount of Compound A, or pharmaceutically acceptable salt thereof, corresponding to an amount of about 80 mg of Compound A, as its free base.

134. The method of claim 131, wherein the tablet comprises an amount of Compound A as its malate salt of about 28 mg, about 41 mg, about 69 mg, about 83 mg, about 110 mg or about 138 mg.

135. The method of claim 134, wherein the tablet comprises an amount of Compound A as its malate salt of about 110 mg.

136. The method of any one of claims 98-135, wherein Compound A, or pharmaceutically acceptable salt thereof, is administered as one dose one time per day.

137. The method of any one of claims 98-135, wherein Compound A, or pharmaceutically acceptable salt thereof, is divided into multiple doses that are administered one, two, three, or four times per day.

138. The method of any one of claims 98-137, wherein Compound A, or pharmaceutically acceptable salt thereof, is administered to the human patient after the patient has fasted for four hours.

139. The method of any one of claims 98-138, wherein the human patient fasts for two hours after Compound A, or pharmaceutically acceptable salt thereof, is administered to the human patient.

140. The method of any one of claims 98-139, wherein the human patient has relapsed or progressed after administration of at least one prior standard therapy.

141. The method of claim 140, wherein the human patient has biliary tract cancer.

142. The method of claim 141, wherein the human patient has relapsed or progressed after administration of gemcitabine-based chemotherapy.

143. The method of claim 140, wherein the human patient has urothelial cancer.

144. The method of claim 143, wherein the human patient has relapsed or progressed after administration of chemotherapy, an anti-PD1/PDL1 therapy, or a combination of chemotherapy and an anti-PD1/PDL1 therapy.

145. The method of claim 144, wherein the chemotherapy is cisplatin-based or carboplatin-based chemotherapy.

146. The method of claim 140, wherein the human patient has non-small cell lung cancer.

147. The method of claim 146, wherein the human patient has relapsed or progressed after administration of chemotherapy, an anti-PD1/PDL1 therapy, or a combination of chemotherapy and an anti-PD1/PDL1 therapy.
